(19) United States
(12) Patent Application Publication
(10) Pub. No.: US 2025/0082587 A1
(43) Pub. Date: Mar. 13, 2025

(54) Method Of Treating Major Depressive Disorder

(71) Applicant: CLEXIO BIOSCIENCES LTD., Yokne'am (IL)

(72) Inventor: David DANGOOR, Jerusalem (IL)

(21) Appl. No.: 18/882,915

(22) Filed: Sep. 12, 2024

Related U.S. Application Data
(60) Provisional application No. 63/582,081, filed on Sep. 12, 2023.

(51) Int. Cl. A61K 31/135; A61K 45/06; A61P 25/24

(52) U.S. Cl. CPC A61K 31/135; A61K 45/06; A61P 25/24

(57) ABSTRACT

The present disclosure provides methods for safe and efficacious administration of esketamine and pharmaceutically acceptable salts thereof.

### CROSS-REFERENCE TO RELATED APPLICATIONS

The present application claims the benefit of priority to U.S. Provisional App. No. 63/582,081, filed Sep. 12, 2023, the entire contents of which are incorporated herein by reference.

### FIELD OF THE INVENTION

The present disclosure provides methods for the safe and efficacious administration of esketamine and pharmaceutically acceptable salts thereof.

### BACKGROUND TO THE INVENTION

Ketamine is a non-barbiturate, rapid acting, induction and general anesthetic agent that acts primarily via NMDA receptor antagonism in the CNS. The drug has been available in the United States since 1970 under the tradename Ketalar®. In 1971, DE2062620 described ketamine's (−) enantiomer, esketamine. Since 2019, intranasal 56 and 85 mg esketamine has been approved, under the tradename Spravato®, for the treatment of treatment resistant depression.

The results of a Phase II study (NCT04103892) in depressed patients with an inadequate response to existing antidepressant therapy and of a second study (NL6030) in treatment resistant depression have both been published. The first study administered 40 mg once daily oral esketamine for 28 days to a depressed patient population with a baseline MADRS score of at least 24. The second study administered 30 mg three times daily orally for 6 weeks to a depressed population with a baseline HADS-17 score of at least 18, which approximately corresponds to a MADRS score of 24.

A subject's MADRS score is not necessarily diagnostic of Major Depressive Disorder, but MDD subjects will often exhibit elevated scores, that is, MADRS scores higher than 6. More severe presentations of MDD will often be accompanied by much higher MADRS scores.

Despite placebo-controlled, double blind studies of esketamine in treating depression and other conditions having been run for more than a decade, it appears that there has been no attempt made to identify whether baseline depression severity might be useful in selecting the esketamine treatment regimen most likely to provide symptom relief. Surprisingly, it has now been found that oral esketamine is clinically more effective in patients initiating treatment with more severe depression than those with less severe depression.

### SUMMARY OF THE INVENTION

In one embodiment, the invention relates to methods of treating major depressive disorder (MDD) in a subject in need thereof comprising determining or having determined said subject's Montgomery-Åsberg Depression Rating Scale (MADRS) score, and orally administering an immediate release oral dosage form comprising 60 mg of esketamine once daily for at least 28 days to the subject that is determined to have a Montgomery-Åsberg Depression Rating Scale (MADRS) score of at least 28.

### DESCRIPTION OF THE INVENTION

The present inventions may be understood more readily by reference to the following detailed description taken in connection with the accompanying examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a particle” is a reference to one or more of such particles and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.” The phrase “at least about x” is intended to embrace both “about x” and “at least x”. It is also understood that where a parameter range is provided, all integers within that range, and tenths thereof, are also provided by the invention. For example, “2-5 hours” includes 2 hours, 2.1 hours, 2.2 hours, 2.3 hours etc., up to 5 hours.

“Subject,” as used herein, includes humans. The terms “human,” “patient,” and “subject” are used interchangeably herein.

The present invention is directed to methods of treating major depressive disorder in a subject in need thereof comprising once daily orally administering to said subject an immediate release oral dosage form comprising about 60 mg of esketamine over at least 28 days.

As used herein, the term “major depressive disorder”, or MDD, is characterized as a psychiatric disorder meeting five criteria: 1) the presence during the same 2 week period which together represent a change from previous functioning, of a depressed/sad mood or a loss of interest and pleasure, together with five (or more) of the following additional criteria occurring nearly every day i) depressed/sad mood ii) loss of interest and pleasure iii) significant weight loss when not dieting or weight gain or a decrease or increase in appetite iv) insomnia or hypersomnia v) psychomotor agitation or retardation vi) fatigue or loss of energy vii) feelings of worthlessness or excessive or inappropriate guilt viii) diminished ability to think or concentrate or indecisiveness ix) recurrent thoughts of death or suicidal ideation, planning or attempt: 2) the symptoms cause clinically significant distress or impairment in social, occupational or other functioning: 3) the episode is not better accounted for by a psychotic disorder: 4) the episode is not attributable to the physiological effects of a substance or to another medical condition: 5) there has never been a manic or hypomanic episode (Diagnostic and Statistical Manual of Mental Disorders, 5th Edition, American Psychiatric Association, 2013). Other indications contemplated include treating, preventing, or ameliorating one or more symptoms of a disorder including, but not limited to depression, refractory depression, suicidality and depression associated with genetic disorders.

In one embodiment of the present invention, the major depressive disorder is with anxious distress. In another embodiment, the disorder is with mixed features. In another embodiment of the present invention, the disorder is with melancholic features. In another embodiment of the present invention, the disorder is with atypical features. In another embodiment of the present invention, the disorder is with mood-congruent psychotic features. In another embodiment of the present invention, the disorder is with mood-incongruent psychotic features. In another embodiment of the present invention, the disorder is with catatonia. In another embodiment of the present invention, the disorder is with peripartum onset. In another embodiment of the present invention, the disorder is with seasonal pattern.

In some aspects, the major depressive disorder is moderate-to-severe major depressive disorder. In some aspects, the major depressive disorder is moderate major depressive disorder. the major depressive disorder is severe major depressive disorder.

In one embodiment of the present invention, the major depressive disorder has not responded to adequate doses and treatment duration of antidepressants other than ketamine or esketamine. In some aspects, the non-responder has failed to demonstrate an improvement of up to 25% in MADRS score, or a similar psychometric score, after adequate doses and treatment duration of antidepressants other than ketamine or esketamine. In other aspects, the non-responder has demonstrated an incomplete improvement of between 25-50% in MADRS score, or a similar psychometric score, after adequate doses and treatment duration of antidepressants other than ketamine or esketamine. In other aspects, the non-responder has demonstrated an inadequate improvement of up to 50% in MADRS score, or a similar psychometric score, after adequate doses and treatment duration of antidepressants other than ketamine or esketamine. In some aspects, the adequate doses and treatment duration of antidepressants other than ketamine or esketamine, refers to doses and treatment duration of one, or more, antidepressants other than ketamine or esketamine during the current depressive episode. In other aspects, the adequate course refers to the non-response to doses and treatment duration of one, or more, antidepressants other than ketamine or esketamine during a previous depressive episode. In other aspects, the adequate course refers to the non-response to doses and treatment duration of one, or more, antidepressants other than ketamine or esketamine both during a previous depressive episode and during the current depressive episode. In some aspects, the disorder is treatment-refractory or treatment-resistant depression, i.e., depression that has failed to respond to adequate doses and treatment duration of at least two antidepressants other than ketamine or esketamine.

As used herein, the term “treating major depressive disorder” can refer to a reduction of the symptoms of Major Depressive Disorder, as measured by reduction in the Montgomery-Åsberg Depression Rating Scale (MADRS) score. Reduction in the MADRS score is understood by those of skill in the art to be an improvement in the MADRS score. In some aspects, the term “treating major depressive disorder” refers to a change from baseline, as measured the MADRS score. In some aspects, the term “treating major depressive disorder” refers to a remission, as measured by reduction in the MADRS score. In some aspects, the term “treating major depressive disorder” refers to a 50% or greater improvement, as measured the MADRS score.

In other aspects, the term “treating major depressive disorder” refers to change from baseline on the physician-administered Global Impression Severity rating scale (CGI-S).

In other aspects, the term “treating major depressive disorder” refers to change from baseline on the physician-administered Global Impression Improvement rating scale (CGI-I).

In other aspects, the term “treating major depressive disorder” refers to change from baseline on the Quick Inventory of Depressive Symptomatology rating scale (QIDS-SR16).

In one embodiment of the present invention, said increases, decreases and reductions of scores and/or presentations of symptoms can be assessed in comparison to the scores and/or presentations of symptoms as identified before treatment is initiated, i.e., baseline. In another embodiment of the present invention, said increases, decreases and reductions of scores and/or presentations of symptoms can be assessed in comparison to the scores and/or presentations of symptoms as identified in a different treatment group, for example, a control population. In another embodiment of the present invention, said increases, decreases and reductions of scores and/or presentations of symptoms of a population of subjects can be assessed in comparison to the scores and/or presentations of symptoms as identified in a different treatment group, for example, a control population of subjects. In some aspects, a population of subjects includes at least about 2, 3, 4, 5, 10, 20, 30, 40, 50, 60, 70, 80, 90, 100, 125, 150, 175, 200, 250, or about 300 subjects.

In one embodiment of the present invention, the different treatment group is a second medication other than ketamine or esketamine. In another embodiment, the different treatment group is a placebo. In another embodiment of the present invention, said increases, decreases and reductions of scores and/or presentations of symptoms can be assessed in comparison to the scores and/or presentations of symptoms as identified in a different treatment group and also in comparison to the scores and/or presentations of symptoms as identified before treatment is initiated, i.e., baseline, for example, the change from baseline in comparison to placebo.

In one embodiment of the present invention, the reduction of the symptoms of major depressive disorder are on or after at least 7 days of treatment with esketamine. In another embodiment of the present invention, the reduction of the symptoms of major depressive disorder are on or after at least 14 days of treatment with esketamine. In another embodiment of the present invention, the reduction of the symptoms of major depressive disorder are on or after at least 21 days of treatment with esketamine. In another embodiment of the present invention, the reduction of the symptoms of major depressive disorder are on or after 28 days of treatment with esketamine. In another embodiment of the present invention, the reduction of the symptoms of major depressive disorder are on or after 30 days of treatment regimen with esketamine. In another embodiment of the present invention, the reduction of the symptoms of major depressive disorder are on or after 90 days of treatment with esketamine. In another embodiment of the present invention, the reduction of the symptoms of major depressive disorder are on or after 180 days of treatment with esketamine. In another embodiment of the present invention, the reduction of the symptoms of major depressive disorder are on or after 365 days of treatment with esketamine. In another embodiment of the present invention, the reduction of the symptoms of major depressive disorder are on or after 730 days of treatment with esketamine. In another embodiment of the present invention, the treatment with esketamine results in a reduction of the symptoms of major depressive disorder after at least 28 days of treatment. In another embodiment of the present invention, the treatment with esketamine results in a reduction of the symptoms of major depressive disorder after at least 90 days of treatment. In another embodiment of the present invention, the treatment with esketamine results in a reduction of the symptoms of major depressive disorder after at least 180 days of treatment. In another embodiment of the present invention, the treatment with esketamine results in a reduction of the symptoms of major depressive disorder after at least 365 days of treatment. In another embodiment of the present invention, the treatment with esketamine results in a reduction of the symptoms of major depressive disorder after at least 730 days of treatment.

In one embodiment, the reduction of the symptoms of major depressive disorder are on or after 28 days of treatment with esketamine and are in comparison to the scores and/or presentations of symptoms as identified before treatment is initiated, i.e. baseline. In another embodiment, the reduction of the symptoms of major depressive disorder are on or after 28 days of treatment with esketamine and are in comparison to the scores and/or presentations of symptoms as identified in a different treatment group. In one embodiment, the different treatment group is a second medication other than ketamine or esketamine. In another embodiment, the different treatment group is a placebo. In one embodiment, the reduction of the symptoms of major depressive disorder are on or after 28 days of treatment with esketamine and are in comparison to the scores and/or presentations of symptoms as identified in a placebo and are in comparison to the scores and/or presentations of symptoms as identified before treatment is initiated, i.e. baseline, for example, the change from baseline in comparison to placebo.

The methods of the disclosure will exhibit an acceptable safety and/or tolerability profile. That is, the benefits achieved using the methods of the disclosure will outweigh any safety and/or tolerability considerations exhibited by using the disclosed methods, as compared to placebo. In other aspects, the benefits achieved using the methods of the disclosure will outweigh any safety and/or tolerability considerations exhibited by using the disclosed methods, as compared to other methods of treating MDD, including treatment-resistant MDD. Other methods of treating MDD, including treatment-resistant MDD include other methods of using ketamine and esketamine. For example, the benefits achieved using the methods of the disclosure will outweigh any adverse events including, for example, untoward changes in hematology, biochemistry, urinalysis, immunological parameters, physical examination findings, blood pressure, and/or heart rate, as compared to placebo. In other aspects, the benefits achieved using the methods of the disclosure will outweigh any adverse events including, for example, changes in hematology, biochemistry, urinalysis, immunological parameters, physical examination findings, blood pressure, and/or heart rate, as compared to other methods of treating MDD, including treatment-resistant MDD.

In other aspects, the benefits achieved using the methods of the disclosure with outweigh any adverse events in 12 lead ECG findings, method discontinuation, Digit Symbol Substitution Test (DSST), reaction time test (Cambridge COGNITION and/or Cogstate battery), self-administered Stanford sleepiness scale, a Bladder Pain/Interstitial Cystitis Symptom Score (BPIC-SS), a Modified Observer's Alertness/Sedation Scale (MOAA/S), a Clinician-Administered Dissociative States Scale (CADSS), a Suicidality Scale-Clinician-Rated Columbia Suicide Severity Rating Scale (C-SSRS), 4 items positive symptoms subscale from the Brief Psychiatric Rating Scale (BPRS), and/or 20 item Physician Withdrawal Checklist (PWC-20), as compared to placebo. In other aspects, the benefits achieved using the methods of the disclosure with outweigh any adverse events in 12 lead ECG findings, method discontinuation, Digit Symbol Substitution Test (DSST), reaction time test (Cambridge COGNITION and/or Cogstate battery), self-administered Stanford sleepiness scale, a Bladder Pain/Interstitial Cystitis Symptom Score (BPIC-SS), a Modified Observer's Alertness/Sedation Scale (MOAA/S), a Clinician-Administered Dissociative States Scale (CADSS), a Suicidality Scale-Clinician-Rated Columbia Suicide Severity Rating Scale (C-SSRS), 4 items positive symptoms subscale from the Brief Psychiatric Rating Scale (BPRS), and/or 20 item Physician Withdrawal Checklist (PWC-20), as compared to other methods of treating MDD, including treatment-resistant MDD.

As used herein, the term “ketamine” shall refer to the chemical compound dl 2-(2-chlorophenyl)-2 (methylamino)cyclohexanone, or a pharmaceutically acceptable salt thereof.

As used herein, the term “esketamine” shall refer to the(S)-enantiomer of ketamine also known as the chemical compound (2S)-2-(2-Chlorophenyl)-2-(methylamino)cyclohexanone, or a pharmaceutically acceptable salt thereof. As used herein, the term “esketamine” shall be understood to be to the exclusion of the compound as found, without an enantiomeric excess, in ketamine, or a pharmaceutically acceptable salt thereof, i.e., the use herein of the term “esketamine” shall be understood to not relate to the enantiomer as present within ketamine, or a pharmaceutically acceptable salt thereof. In one embodiment, the esketamine, or a pharmaceutically acceptable salt thereof, is the hydrochloride salt of esketamine, i.e., esketamine hydrochloride.

As used herein, the term “(R)-ketamine” shall refer to the (R)-enantiomer of ketamine also known as the chemical compound (2R)-2-(2-Chlorophenyl)-2-(methylamino) cyclohexanone, or a pharmaceutically acceptable salt thereof. As used herein, the term “(R)-ketamine” shall be understood to be to the exclusion of the compound as found, without an enantiomeric excess, in ketamine, or a pharmaceutically acceptable salt thereof.

The chemical compounds described herein according to the invention are also intended to include such compounds wherein the molecular structures include isotopes of carbon, hydrogen and nitrogen atoms occurring on those structures. Isotopes include those atoms having the same atomic number but different mass numbers. For example, isotopes of hydrogen include deuterium. Isotopes of carbon include ¹³C. Isotopes of nitrogen include ¹⁵N.

Accordingly, within the chemical structure of any chemical compound taught in this application as suitable for the formulations disclosed herein:
any hydrogen atom or group of hydrogen atoms, could suitably be replaced by an isotope of hydrogen, i.e., deuterium;
any carbon atom or group of carbon atoms, could suitably be replaced by an isotope of carbon, i.e., ¹³C; and
any nitrogen atom or group of nitrogen atoms, could suitably be replaced by an isotope of nitrogen, i.e., ¹⁵N.

In some aspects, oral dosage forms of the disclosure include esketamine, i.e., esketamine as a free base. In other aspects, oral dosage forms of the disclosure include pharmaceutically acceptable salts of esketamine. As used herein, amounts of esketamine present in the oral dosage forms of the disclosure refer to amounts of esketamine free base. For example, in those aspects wherein the oral dosage form comprises esketamine free base, “60 mg of esketamine” refers to 60 mg of the esketamine free base in the oral dosage form. In aspects wherein the oral dosage form comprises a pharmaceutically acceptable salt of esketamine, such as esketamine hydrochloride, “60 mg of esketamine” refers to 60 mg esketamine free base, based on 69.18 mg of esketamine hydrochloride in the oral dosage form.

As used herein, the term “immediate release oral dosage form” can refer to a dosage form that upon oral ingestion by a human releases substantially all of a contained active pharmaceutical ingredient (API) into a gastrointestinal tract for biological uptake in a short time. In vitro methods of measuring a release profile of a dosage form, for the purpose of determining whether a dosage form exhibits an immediate release or extended release dissolution profile, are known in the pharmaceutical arts. By such methods, examples of immediate release dosage forms as described herein can be measured to be capable of releasing substantially all of a total amount of at least one type of active pharmaceutical ingredient contained in the dosage form (e.g., at least 75, 80, or 90 weight percent of the total amount of the API in a dosage form) into a solution (e.g., acidic aqueous solution) of a suitable pH within 240 minutes, e.g., in less than 180 minutes, less than 90 minutes, or less than 60, 30, 15, or 5 minutes. For example, a release profile of a dosage form of the present description may be measured by a method that exposes the dosage form to a volume of up to 900 milliliters (e.g., 300 milliliters, or 900 milliliters, based on various test methods) of hydrochloric acid (0.01 to 0.1N) (e.g., aqueous hydrochloric acid) at a pH of from 1 to 2, and at a temperature of 37 degrees Celsius.

In one embodiment of the invention, the immediate release oral dosage form is a liquid preparation such as a suspension, elixir, or solution. In another embodiment of the invention, the immediate release oral dosage forms are solid preparations, for example, powders, capsules, caplets, gelcaps, and tablets. In a preferred embodiment, the immediate release oral dosage form is a tablet, gelcap, or capsule. In a more preferred embodiment, the immediate release oral dosage form is a tablet.

To prepare the preparations, i.e., the immediate release oral dosage forms, of this invention, esketamine, and optionally, at least one second medication other than (R)-ketamine, are admixed with pharmaceutical carriers according to conventional pharmaceutical compounding techniques, which carriers may take a wide variety of forms depending of the form of preparation desired for administration. In preparing the oral preparations, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like. For solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are employed.

In one embodiment of invention, the method comprises the treating of major depressive disorder in a subject in need thereof by the administration to said patient of an immediate release oral dosage form comprising about 60 mg of esketamine, preferably 60 mg of esketamine, wherein the oral dosage form is an abuse deterrent formulation. In a more preferred embodiment, the abuse deterrent formulation is a tablet. Abuse deterrent tablet formulations can be prepared by methods known in the art including as found in U.S. Pat. No. 7,955,619, WO2014006004, WO2008033523, WO2008023261, WO2016094358, WO2020225773 and US2004052731 each of which is hereby incorporated by reference.

The present invention is further directed to methods of treating major depressive disorder in a subject in need thereof comprising once daily orally administering to said subject an immediate release oral dosage form comprising about 60 mg of esketamine over at least 28 days wherein, prior to administration of the esketamine, the subject is determined to have a Montgomery-Åsberg Depression Rating Scale (MADRS) score of at least 28.

As use herein, the term “Montgomery-Åsberg Depression Rating Scale” is synonymous with the term MADRS and refers to the widely used, 10-item clinician-rated measure of depressive severity as designed by Montgomery and Åsberg (Br J Psychiatry 1979 April; 134:382-9). The MADRS is typically administrable to adults of 18 years and over and is rated based on a clinical interview with the patient. 9 of the 10 items rated are based upon patient report, whilst the tenth is based upon observation by the administrator during the rating interview. Each of the 10 items evaluates core symptoms of depression and has 4 defined scale steps (scored 0, 2, 4 and 6 points) and 3 in-between steps (scored 1, 3 and 5) denoted as “Worsening symptoms”.

Early attempts to stratify MADRS scores to disease severity were based around its potential correlation to other depression related scores which already had preset grades. Later attempts have focused less on the correlation to other scores and more on the actual clinical presentation of patients at the identified MADRS score.

As used herein, the term ‘moderate-to-severe major depressive disorder (MDD)’ can refer to a subject with a MADRS score of at least 28.

As used herein, the term ‘moderate major depressive disorder (MDD)’ can refer to a subject with a MADRS score of at least 28 and less than 33.

As used herein, the term ‘severe major depressive disorder (MDD)’ can refer to a subject with a MADRS score of at least 33.

In one embodiment of the present invention, the method of treating major depressive disorder in a subject in need thereof comprises orally administering once daily to the subject an immediate release oral dosage form comprising about 60 mg of esketamine over at least 28 days wherein, prior to administration of the esketamine, the subject is determined to have a Montgomery-Åsberg Depression Rating Scale (MADRS) score of at least 28.

As used herein, the term ‘determining’ is synonymous with other language and terms, such as identified as having, rated as being, known to have etc., and can refer to the clinician's assessment of the subject's MADRS score according to the established test protocols.

According to the invention, initiation of an esketamine treatment method as described herein is determined based on the subject's MADRS score. That is, a subject exhibiting a MADRS score at or above 28 can begin treatment as soon as practical, which can be assessed by one of skill in the art. In one embodiment of the present invention, the determination of the subject's MADRS score is at the stage of clinical trial patient screening. In another aspect of the invention, the determination of the subject's MADRS score is prior to initiation of an esketamine treatment method as described herein, i.e., baseline. In some aspects, the subject's MADRS score is determined the same day that an esketamine treatment method of the disclosure is initiated. In some aspects, the subject's MADRS score is determined within one week of an esketamine treatment method of the disclosure being initiated. In some aspects, the subject's MADRS score is determined within two weeks of an esketamine treatment method of the disclosure being initiated. In some aspects, the subject's MADRS score is determined within three weeks of an esketamine treatment method of the disclosure being initiated. In some aspects, the subject's MADRS score is determined within four weeks of an esketamine treatment method of the disclosure being initiated.

In one embodiment of the present invention, the method of treating major depressive disorder (MDD) in a subject in need thereof comprises determining or having determined said subject's Montgomery-Åsberg Depression Rating Scale (MADRS) score, and orally administering an immediate release oral dosage form comprising 60 mg of esketamine once daily for at least 28 days to the subject that is determined to have a Montgomery-Åsberg Depression Rating Scale (MADRS) score of at least 28. In another embodiment of the present invention, the method of treating major depressive disorder (MDD) in a subject in need thereof comprises determining said subject's Montgomery-Åsberg Depression Rating Scale (MADRS) score, and orally administering an immediate release oral dosage form comprising 60 mg of esketamine once daily for at least 28 days to the subject that is determined to have a Montgomery-Åsberg Depression Rating Scale (MADRS) score of at least 28. In another embodiment of the present invention, the method of treating major depressive disorder (MDD) in a subject in need thereof comprises having determined said subject's Montgomery-Åsberg Depression Rating Scale (MADRS) score, orally administering an immediate release oral dosage form comprising 60 mg of esketamine once daily for at least 28 days to the subject that is determined to have a Montgomery-Åsberg Depression Rating Scale (MADRS) score of at least 28.

In one embodiment of the present invention, the method of treating major depressive disorder (MDD) in a subject in need thereof comprises determining or having determined said subject's Montgomery-Åsberg Depression Rating Scale (MADRS) score immediately prior to initiation of esketamine treatment, and initiating the oral administration of an immediate release oral dosage form comprising 60 mg of esketamine once daily for at least 28 days to the subject that is determined immediately prior to initiation of esketamine treatment to have a Montgomery-Åsberg Depression Rating Scale (MADRS) score of at least 28. In another embodiment of the present invention, the method of treating major depressive disorder (MDD) in a subject in need thereof comprises determining said subject's Montgomery-Åsberg Depression Rating Scale (MADRS) score immediately prior to initiation of esketamine treatment, and initiating the oral administration of an immediate release oral dosage form comprising 60 mg of esketamine once daily for at least 28 days to the subject that is determined immediately prior to initiation of esketamine treatment to have a Montgomery-Åsberg Depression Rating Scale (MADRS) score of at least 28. In another embodiment of the present invention, the method of treating major depressive disorder (MDD) in a subject in need thereof comprises having determined said subject's Montgomery-Åsberg Depression Rating Scale (MADRS) score immediately prior to initiation of esketamine treatment, initiating the oral administration of an immediate release oral dosage form comprising 60 mg of esketamine once daily for at least 28 days to the subject that is determined immediately prior to initiation of esketamine treatment to have a Montgomery-Åsberg Depression Rating Scale (MADRS) score of at least 28.

In one embodiment of the present invention, the method of treating major depressive disorder (MDD) in a subject in need thereof comprises determining or having determined whether said subject's Montgomery-Åsberg Depression Rating Scale (MADRS) score is at least 28, and orally administering an immediate release oral dosage form comprising 60 mg of esketamine once daily for at least 28 days to the subject that is determined to have a Montgomery-Åsberg Depression Rating Scale (MADRS) score of at least 28. In another embodiment of the present invention, the method of treating major depressive disorder (MDD) in a subject in need thereof comprises determining whether said subject's Montgomery-Åsberg Depression Rating Scale (MADRS) score is at least 28, and orally administering an immediate release oral dosage form comprising 60 mg of esketamine once daily for at least 28 days to the subject that is determined to have a Montgomery-Åsberg Depression Rating Scale (MADRS) score of at least 28. In another embodiment of the present invention, the method of treating major depressive disorder (MDD) in a subject in need thereof comprises having determined whether said subject's Montgomery-Åsberg Depression Rating Scale (MADRS) score is at least 28, orally administering an immediate release oral dosage form comprising 60 mg of esketamine once daily for at least 28 days to the subject that is determined to have a Montgomery-Åsberg Depression Rating Scale (MADRS) score of at least 28.

In one embodiment of the present invention, the method of treating major depressive disorder (MDD) in a subject in need thereof comprises orally administering once daily for at least 28 days to said subject an immediate release oral dosage form comprising 60 mg of esketamine or a pharmaceutically acceptable salt thereof, wherein, prior to administration, the subject is known to have a Montgomery-Åsberg Depression Rating Scale (MADRS) score of at least 28.

One embodiment of the present invention is directed to reducing from baseline, a human's Montgomery-Åsberg Depression Rating Scale (MADRS) score after at least 28 days when compared to placebo. In these methods, the human is diagnosed as having major depressive disorder (MDD) and is determined to have a MADRS score of at least 28. According to the invention, the human is orally administered an immediate release oral dosage form comprising 60 mg of esketamine or a pharmaceutically acceptable salt thereof, once daily for at least 28 days.

Another embodiment of the present invention is directed to methods of major depressive disorder (MDD) in a human in need thereof by reducing from baseline, the human's Montgomery-Åsberg Depression Rating Scale (MADRS) score after at least 28 days when compared to placebo. These methods are useful in treating humans determined to have a MADRS score of at least 28 prior to the initiation of a disclosed treatment. According to these methods, the human is orally administered an immediate release oral dosage form comprising 60 mg of esketamine or a pharmaceutically acceptable salt thereof, once daily for at least 28 days.

Another embodiment of the invention is directed to methods of treating a population of humans having major depressive disorder (MDD), in particular, a population of humans having moderate-to-severe MDD. In these aspects, the Montgomery-Åsberg Depression Rating Scale (MADRS) score of each human in the population having moderate-to-severe MDD is determined to be at least 28. In other aspects, the MADRS score of each human in the population having moderate-to-severe MDD has been determined to be at least 28. Each human in the population of is treated by orally administering an immediate release oral dosage form comprising 60 mg of esketamine or a pharmaceutically acceptable salt thereof, once daily for at least 28 days. According to these methods, the treated population of humans demonstrates a statistically significant reduction in MADRS score at at least day 28, as compared to a control population of humans that was not treated with the immediate release oral dosage form comprising 60 mg of esketamine or a pharmaceutically acceptable salt thereof, once daily for at least 28 days. In some aspects, statistical significance is defined as a one-sided p value of ≤0.05. Examples of inclusion and exclusion criteria for the treated population, as well as the control population, are further described herein.

In one embodiment of the present invention, the method of treating major depressive disorder (MDD) in a subject in need thereof comprises determining or having determined said subject's Montgomery-Åsberg Depression Rating Scale (MADRS) score, and orally administering an immediate release oral dosage form comprising 60 mg of esketamine to the subject that is determined to have a Montgomery-Åsberg Depression Rating Scale (MADRS) score of at least 28 and wherein the administration of the esketamine is once daily for at least 28 days. In another embodiment of the present invention, the method of treating major depressive disorder (MDD) in a subject in need thereof comprises determining said subject's Montgomery-Åsberg Depression Rating Scale (MADRS) score, and orally administering an immediate release oral dosage form comprising 60 mg of esketamine to the subject that is determined to have a Montgomery-Åsberg Depression Rating Scale (MADRS) score of at least 28 and wherein the administration of the esketamine is once daily for at least 28 days. In another embodiment of the present invention, the method of treating major depressive disorder (MDD) in a subject in need thereof comprises having determined said subject's Montgomery-Åsberg Depression Rating Scale (MADRS) score, orally administering an immediate release oral dosage form comprising 60 mg of esketamine to the subject that is determined to have a Montgomery-Åsberg Depression Rating Scale (MADRS) score of at least 28 and wherein the administration of the esketamine is once daily for at least 28 days.

The present invention is further directed to a method of treating major depressive disorder in a human patient in need thereof by the oral administration to said patient of an immediate release oral dosage form comprising about 60 mg of esketamine, preferably 60 mg esketamine, over a treatment regimen of at least 28 days wherein the administration is self-administered. As used herein “self-administered” refers to administration wherein the patient is responsible for taking the medication and is not assisted during the oral administration of the immediate release oral dosage form by a healthcare professional. In some aspects, one or more of the administrations may be assisted by a healthcare professional and one or more of the administration may be self-administered over the treatment regimen. In one embodiment, said self-administration is in the patient's own home. In a preferred embodiment, said self-administration is at night. In a more preferred embodiment, said self-administration is before the patient goes to sleep.

In another embodiment, the patient has no restrictions on driving in the 24 hours immediately following the oral administration of the immediate release oral dosage form comprising about between about 60 mg of esketamine, preferably 60 mg of esketamine. That is, the oral administration of the immediate release oral dosage form does not result in a mental or motor impairment that negatively affects the patient's ability to operative a motor vehicle. In the 24 hours immediately following the administration.

In yet another embodiment, the patient is restricted from driving for no more than 10 hours after the oral administration of the immediate release oral dosage form comprising about 60 mg of esketamine, preferably 60 mg of esketamine. In a preferred embodiment, the patient is restricted from driving for no more than 8 hours after the administration. In another preferred embodiment, the patient is restricted from driving for no more than 6 hours after the administration. In a more preferred embodiment, the patient is restricted from driving for no more than 2 hours after the administration. In a most preferred embodiment, the patient is restricted from driving for no more than an hour after the oral administration of the immediate release oral dosage form comprising about 60 mg of esketamine, preferably 60 mg of esketamine.

The present invention is further directed to methods of treating major depressive disorder in a human patient in need thereof by the oral administration to said patient of an immediate release oral dosage form comprising about 60 mg of esketamine, preferably 60 mg of esketamine, further comprising the administration of a second medication other than (R)-ketamine.

In a preferred embodiment, the second medication is an antidepressant, an antimanic agent or an anxiolytic drug. In one embodiment of the invention, the antidepressant is selected from the group consisting of mono-amine oxidase inhibitors (MAOI), tricyclic antidepressants (TCA), serotonin specific reuptake inhibitors (SSRI), serotonin noradrenergic reuptake inhibitors (SNRI), noradrenaline reuptake inhibitor (NRI), “natural products” (such as Kava-Kava, St. John's Wort), dietary supplement (such as s-adenosylmethionine) and others. More specifically, antidepressants include, but are not limited to, imipramine, amitriptyline, desipramine, nortriptyline, doxepin, protriptyline, trimipramine, maprotiline, amoxapine, trazodone, bupropion, clomipramine, fluoxetine, citalopram, escitalopram, sertraline, paroxetine, tianeptine, agomelatine, nefazadone, venlafaxine, desvenlafaxine, vilazodone, vortioxetine, duloxetine, reboxetine, mirtazapine, mianserin, phenelzine, tranylcypromine, and/or moclobemide.

In another preferred embodiment, the second medication is an antimanic agent. In one embodiment of the invention, the antimanic agent is selected from the group consisting of carbamazepine, gabapentin, Lithium or a pharmaceutically acceptable salt thereof, valproic acid and antipsychotic medications such as lurasidone, cariprazine, olanzapine, risperidone, quetiapine, paliperidone, aripiprazole and brexpiprazole.

In another preferred embodiment, the second medication is an anxiolytic drug. In one embodiment of the invention, the anxiolytic drug is selected from the group consisting of Alprazolam, Bromazepam, Chlordiazepoxide, Clonazepam, Clorazepate, Diazepam, Flurazepam, Lorazepam, Oxazepam, Temazepam, Triazolam, Buspirone, Gepirone, Ipsapirone, Hydroxyzine, Amobarbital, Pentobarbital, Phenobarbital, Thiopental and Propanolol.

The present invention is further directed to a method of treating major depressive disorder in a human patient in need thereof, by the oral administration to said patient of an immediate release oral dosage form comprising about 60 mg of esketamine, preferably 60 mg of esketamine and an antidepressant selected from the group consisting of fluoxetine, sertraline, paroxetine, citalopram, escitalopram, venlafaxine, desvenlafaxine, duloxetine, and fluvoxamine and wherein the administration is daily over a treatment regimen of at least 28 days.

This invention will be better understood by reference to the Examples, which follow, but those skilled in the art will readily appreciate that the specific experiments detailed are only illustrative of the invention as described more fully in the claims which follow thereafter.

### EXAMPLES

### Example 1: Immediate Release Oral Dosage Forms

Oral dosage forms of esketamine hydrochloride are manufactured according to the procedures described in WO2016094358, which is incorporated in its entirety, herein, by reference. The formulation is presented in Table 1.

TABLE 1

| | 60 mg tablet |
|---|---|
| | Ingredient (% (w/w)) |
| 32.0% Coated Esketamine Granules | 18.75 |
| Coated Polymer Granules | 10.04 |
| Mannitol | 33.01 |
| Crospovidone | 20.00 |
| Microcrystalline Cellulose | 13.00 |
| Carbomer | 2.00 |
| Sodium Bicarbonate | 2.00 |
| Colloidal silicon Dioxide | 0.20 |
| Magnesium stearate | 1.00 |

### Example 2

A Phase 2, randomized double blind study to assess the safety and efficacy of 40 mg immediate release once daily esketamine as an adjunct therapy for subjects with MDD with an inadequate response to standard antidepressant therapy.

The safety and efficacy of 65 subjects with MDD with an inadequate response to standard antidepressant therapy were assessed in a Phase 2, randomized double blind study following the administration of 40 mg of immediate release once daily esketamine as adjunct therapy.

The study comprised 3 phases, screening (Days 0-28), double-blind treatment (days 29-56) and post-treatment safety follow-up (days 57-70) following the last study treatment administration.

The study's primary efficacy endpoint was the change from baseline to week 4 in the 10 item Montgomery-Åsberg Depression Rating Scale (MADRS). Key secondary efficacy endpoints included the change in the self-rated Symptoms of Depression Questionnaire (SDQ) from Baseline to Week 4, the change in the Sheehan Disability Scale (SDS) from Baseline to Week 4, the change in the MADRS score from Baseline to Week 2 and the change in the Clinical Global Impression-Severity (CGI-S) from Baseline to Week 4.

During screening, subjects were assessed for study eligibility and washed out from disallowed drugs. Inclusion criteria for the study included the subjects presenting with a baseline MADRS of at least 24.

After being found eligible, subjects were randomized to receive once daily, either placebo or 40 mg of oral, immediate release, esketamine, respectively.

Subjects received the first dose of their study drug at the study site and were then closely monitored for 3 hours to assess for potential neuropsychiatric adverse events using a comprehensive set of scales to identify sleepiness, sedation and dissociative effects. Thereafter, the subject was provided with a 1 week supply of the study drug for administration at their place of residence and instructed to take the study drug in the evening (except at the day of the weekly visit when it is taken at the study site) and not to drive for at least 8 hours following the dosing. At every subject visit, a psychiatrist evaluated the subject's MADRS score.

Analysis of the results of subjects who completed the study showed that the primary efficacy endpoint was clinically meaningful and statistically significant between treatment groups. The LS mean of the change in MADRS score from Baseline to Week 4 (Day 29) was-12.96 in the esketamine group and −7.68 in the placebo group while the LS mean difference in the change in MADRS score from baseline to week 4 between the esketamine group and the placebo group was-5.28 and reached statistical significance (p=0.03).

Further analysis of these results was undertaken to review the clinical effect according to the subject's baseline MADRS score. The results of this sub-analysis are presented in Table 2.

TABLE 2

| Baseline MADRS score | <28 | ≥28 | ≥33 |
|---|---|---|---|
| Change from baseline against placebo | +4.25 | −7.425 | −5.978 |
| p value | | 0.005 | 0.050 |

These results suggest that there was no identifiable improvement in depression symptoms in patients treated with 40 mg of immediate release oral esketamine for 28 days and who started treatment with a baseline MADRS of less than 28. In contrast, there was a statistically significant improvement in the symptoms of those patients treated with esketamine who started the study with a baseline MADRS of 28 or more.

### Example 3

A randomized, double-blind, placebo-controlled, multicenter, parallel-design, phase 2 study to assess the efficacy and safety of once-daily 60 mg immediate release oral esketamine for the treatment of adults with major depressive disorder and inadequate response to at least two antidepressants.

A phase II, dose range finding, multinational, double-randomized, double-blind, placebo-controlled study compares the efficacy, safety and tolerability of once daily 60 mg immediate release oral esketamine to placebo treatment in 204 MDD subjects with inadequate response to antidepressant therapy. All subjects remain on their current anti-depressant with no dose change during the study.

The study comprises 3 phases, screening (Days 0-28), double-blind treatment (days 29-56) composed of two 2-week periods (period 1, period 2) and post-treatment safety follow-up (days 57-70) following the last study treatment administration.

During screening, subjects are assessed for study eligibility and washed out from disallowed drugs. Inclusion criteria for the study include the subjects presenting with a baseline MADRS of at least 24. After being found eligible, subjects are randomized to receive, once daily, either placebo or 60 mg immediate release oral esketamine, respectively.

Subjects receive the first dose of their study drug at the study site and are then closely monitored for 3 hours to assess for potential neuropsychiatric adverse events using a comprehensive set of scales to identify sleepiness, sedation and dissociative effects. Thereafter, the subject is provided with a 1 week supply of the study drug for administration at their place of residence and instructed to take the study drug in the evening (except at the day of the weekly visit when it is taken at the study site) and not to drive until the next morning. At every subject visit, a psychiatrist evaluates the subject's MADRS score.

The study's primary efficacy endpoint is the change from baseline to week 4 in the 10 items Montgomery-Åsberg Depression Rating Scale (MADRS).

Secondary efficacy endpoints include the change from baseline Sheehan disability scale (SDS) at 4 weeks, remission rate at 4 weeks (MADRS≤10), responder rate at 4 weeks (≥50% improvement in MADRS), sub-groups analysis of change from baseline to week 4 (in MADRS stratified according to MADRS score at study baseline (<28, ≥28, ≥33), change from baseline in self-rated Symptoms of Depression Questionnaire (SDQ) at 4 weeks, change from baseline in the Quick Inventory of Depressive Symptomatology rating scale (QIDS-SR16), change from baseline in the physician administered Clinical Global Impression Improvement (CGI-I) at 4 weeks and the change from baseline in Physician administered Clinical Global Impression Severity (CGI-S) at 4 weeks.

Exploratory Endpoints include the change from baseline in Generalized Anxiety Disorder 7 items scale (GAD-7) at 4 weeks and the change from baseline to week 2 in Montgomery-Åsberg Depression Rating Scale (MADRS).

Safety and tolerability endpoints include adverse events, hematology, biochemistry and urinalysis, immunological parameters, physical examination findings, blood pressure and heart rate every 30 minutes for the 3 hours following study drug administration, 12 lead ECG findings, withdrawal rates, Digit Symbol Substitution Test (DSST), reaction time test (Cambridge COGNITION), self-administered Stanford sleepiness scale, a Bladder Pain/Interstitial Cystitis Symptom Score (BPIC-SS), a Modified Observer's Alertness/Sedation Scale (MOAA/S), a Clinician-Administered Dissociative States Scale (CADSS), a suicidality scale-Clinician-Rated Columbia Suicide Severity Rating Scale (C-SSRS), 4 items positive symptoms subscale from the Brief Psychiatric Rating Scale (BPRS) and 20 item Physician Withdrawal Checklist (PWC-20) during the follow-up period.

Those skilled in the art will appreciate that numerous changes and modifications can be made to the preferred embodiments of the disclosure and that such changes and modifications can be made without departing from the spirit of the disclosure. It is, therefore, intended that the appended claims cover all such equivalent variations as fall within the true spirit and scope of the disclosure.

The following numbered clauses define various further aspects and features of the present technique:
1. A method of treating major depressive disorder (MDD) in a subject in need thereof comprising:
   a) determining or having determined said subject's Montgomery-Åsberg Depression Rating Scale (MADRS) score, and
   b) orally administering an immediate release oral dosage form comprising 60 mg of esketamine or a pharmaceutically acceptable salt thereof, once daily for at least 28 days to the subject that is determined to have a Montgomery-Åsberg Depression Rating Scale (MADRS) score of at least 28.
2. The method of clause 1, wherein the administration of the oral dosage form is at night.
3. The method of clause 2, wherein the administration of the oral dosage form is before the subject goes to sleep.
4. The method of clause 1, wherein the oral dosage form is self-administered by the patient.
5. The method of clause 4, wherein the self-administration is in the human patient's own home.
6. The method of clause 1, wherein following the administration of the oral dosage form, the patient is restricted from driving for no more than eight hours.
7. The method of clause 1, further comprising the administration of a second medication other than (R)-ketamine.
8. The method of clause 7, wherein the second medication comprises an antidepressant, an antimanic agent or an anxiolytic drug.
9. The method of clause 8, wherein the antidepressant is selected from the group consisting of mono-amine oxidase inhibitors, tricyclic antidepressants, serotonin specific reuptake inhibitors, serotonin noradrenergic reuptake inhibitors and noradrenaline reuptake inhibitors.
10. The method of clause 1, wherein the treatment of major depressive disorder (MDD) comprises an improvement from baseline in the subject's measured Montgomery-Åsberg Depression Rating Scale (MADRS) score after at least 28 days when compared to placebo.
11. The method of clause 1, wherein prior to the administration, the human patient has not responded to adequate doses and treatment durations of antidepressants other than ketamine or esketamine.
12. The method of clause 1, wherein prior to the administration, the human patient has failed to demonstrate an improvement of up to 25% in the MADRS score after adequate doses and treatment durations of antidepressants other than ketamine or esketamine.
13. The method of clause 1, wherein prior to the administration, the human patient has failed to demonstrate an improvement of up to 50% in the MADRS score after adequate doses and treatment durations of antidepressants other than ketamine or esketamine.
14. The method of clause 1, wherein prior to the administration, the human patient has demonstrated an improvement of between 25 and 50% in the MADRS score after adequate doses and treatment durations of antidepressants other than ketamine or esketamine.
15. The method of clause 1, wherein the major depressive disorder (MDD) is moderate-to-severe major depressive disorder (MDD).
16. The method of clause 1, wherein the major depressive disorder (MDD) is moderate major depressive disorder (MDD).
17. The method of clause 1, wherein the major depressive disorder (MDD) is severe major depressive disorder (MDD).
18. The method of clause 1, wherein the esketamine is esketamine hydrochloride.
19. A method of treating major depressive disorder (MDD) in a subject in need thereof comprising:
   a) determining or having determined whether said subject's Montgomery-Åsberg Depression Rating Scale (MADRS) score is at least 28, and
   b) orally administering an immediate release oral dosage form comprising 60 mg of immediate release esketamine or a pharmaceutically acceptable salt thereof, once daily for at least 28 days to the subject that is determined to have a Montgomery-Åsberg Depression Rating Scale (MADRS) score of at least 28.
20. A method of treating major depressive disorder (MDD) in a subject in need thereof comprising:
   a) determining or having determined that said subject's Montgomery-Åsberg Depression Rating Scale (MADRS) score is at least 28, and
   b) orally administering an immediate release oral dosage form comprising 60 mg of immediate release esketamine or a pharmaceutically acceptable salt thereof, once daily for at least 28 days to the subject that is determined to have a Montgomery-Åsberg Depression Rating Scale (MADRS) score of at least 28.
21. A method of treating major depressive disorder (MDD) in a subject in need thereof comprising:
   a) determining or having determined said subject's Montgomery-Åsberg Depression Rating Scale (MADRS) score, and
   b) orally administering an immediate release oral dosage form comprising 60 mg of esketamine or a pharmaceutically acceptable salt thereof, to the subject that is determined to have a Montgomery-Åsberg Depression Rating Scale (MADRS) score of at least 28; and
wherein the administration is once daily for at least 28 days.
22. A method for treating major depressive disorder (MDD) in a subject in need thereof comprising orally administering once daily for at least 28 days to said subject an immediate release oral dosage form comprising 60 mg of esketamine or a pharmaceutically acceptable salt thereof, wherein, prior to administration, the subject is known to have a Montgomery-Åsberg Depression Rating Scale (MADRS) score of at least 28.
23. A method for treating major depressive disorder (MDD) in a subject in need thereof comprising orally administering once daily for at least 28 days to said subject an immediate release oral dosage form comprising 60 mg of esketamine or a pharmaceutically acceptable salt thereof, wherein, prior to administration, the subject is determined to have a Montgomery-Åsberg Depression Rating Scale (MADRS) score of at least 28.
24. A method of reducing from baseline, a human's Montgomery-Åsberg Depression Rating Scale (MADRS) score after at least 28 days when compared to placebo, wherein the human is diagnosed as having major depressive disorder (MDD) and is determined to have a MADRS score of at least 28 comprising orally administering an immediate release oral dosage form comprising 60 mg of esketamine or a pharmaceutically acceptable salt thereof, once daily for at least 28 days to the human.
25. A method of treating major depressive disorder (MDD) in a human in need thereof by reducing from baseline, the human's Montgomery-Åsberg Depression Rating Scale (MADRS) score after at least 28 days when compared to placebo, comprising:
   orally administering an immediate release oral dosage form comprising 60 mg of esketamine or a pharmaceutically acceptable salt thereof, once daily for at least 28 days to the human;
wherein the human is determined to have a MADRS score of at least 28 prior to the initiation of the treatment.
26. A method of treating a population of humans having moderate-to severe MDD comprising
   determining or having determined the Montgomery-Åsberg Depression Rating Scale (MADRS) score of each human in the population of humans having moderate-to severe MDD is at least 28; and
   treating each human in the population by orally administering an immediate release oral dosage form comprising 60 mg of esketamine or a pharmaceutically acceptable salt thereof, once daily for at least 28 days;
   wherein the treated population of humans demonstrates a statistically significant reduction in MADRS score at at least day 28, as compared to a control population of humans that was not treated with the immediate release oral dosage form comprising 60 mg of esketamine or a pharmaceutically acceptable salt thereof, once daily for at least 28 days.

We claim:

1. A method of treating major depressive disorder (MDD) in a subject in need thereof comprising:
a) determining or having determined said subject's Montgomery-Åsberg Depression Rating Scale (MADRS) score, and
b) orally administering an immediate release oral dosage form comprising 60 mg of esketamine or a pharmaceutically acceptable salt thereof, once daily for at least 28 days to the subject that is determined to have a Montgomery-Åsberg Depression Rating Scale (MADRS) score of at least 28.

2. The method of claim 1, wherein the administration of the oral dosage form is at night.

3. The method of claim 2, wherein the administration of the oral dosage form is before the subject goes to sleep.

4. The method of claim 1, wherein the oral dosage form is self-administered by the patient.

5. The method of claim 1, wherein following the administration of the oral dosage form, the patient is restricted from driving for no more than eight hours.

6. The method of claim 1, further comprising the administration of a second medication other than (R)-ketamine.

7. The method of claim 6, wherein the second medication comprises an antidepressant, an antimanic agent or an anxiolytic drug.

8. The method of claim 7, wherein the antidepressant is selected from the group consisting of mono-amine oxidase inhibitors, tricyclic antidepressants, serotonin specific reuptake inhibitors, serotonin noradrenergic reuptake inhibitors and noradrenaline reuptake inhibitors.

9. The method of claim 1, wherein the treatment of major depressive disorder (MDD) comprises an improvement from baseline in the subject's measured Montgomery-Åsberg Depression Rating Scale (MADRS) score after at least 28 days when compared to placebo.

10. The method of claim 1, wherein prior to the administration, the human patient has not responded to adequate doses and treatment durations of antidepressants other than ketamine or esketamine.

11. The method of claim 1, wherein prior to the administration, the human patient has failed to demonstrate an improvement of up to 25% in the MADRS score after adequate doses and treatment durations of antidepressants other than ketamine or esketamine.

12. The method of claim 1, wherein prior to the administration, the human patient has failed to demonstrate an improvement of up to 50% in the MADRS score after adequate doses and treatment durations of antidepressants other than ketamine or esketamine.

13. The method of claim 1, wherein prior to the administration, the human patient has demonstrated an improvement of between 25 and 50% in the MADRS score after adequate doses and treatment durations of antidepressants other than ketamine or esketamine.

14. The method of claim 1, wherein the major depressive disorder (MDD) is moderate-to-severe major depressive disorder (MDD).

15. The method of claim 1, wherein the major depressive disorder (MDD) is moderate major depressive disorder (MDD).

16. The method of claim 1, wherein the major depressive disorder (MDD) is severe major depressive disorder (MDD).

17. The method of claim 1, wherein the esketamine is esketamine hydrochloride.

18. A method of treating major depressive disorder (MDD) in a subject in need thereof comprising:
a) determining or having determined whether said subject's Montgomery-Åsberg Depression Rating Scale (MADRS) score is at least 28, and
b) orally administering an immediate release oral dosage form comprising 60 mg of immediate release esketamine or a pharmaceutically acceptable salt thereof, once daily for at least 28 days to the subject that is determined to have a Montgomery-Åsberg Depression Rating Scale (MADRS) score of at least 28.

19. A method of treating major depressive disorder (MDD) in a subject in need thereof comprising:
a) determining or having determined that said subject's Montgomery-Åsberg Depression Rating Scale (MADRS) score is at least 28, and
b) orally administering an immediate release oral dosage form comprising 60 mg of immediate release esketamine or a pharmaceutically acceptable salt thereof, once daily for at least 28 days to the subject that is determined to have a Montgomery-Åsberg Depression Rating Scale (MADRS) score of at least 28.

20. A method of treating major depressive disorder (MDD) in a subject in need thereof comprising:
a) determining or having determined said subject's Montgomery-Åsberg Depression Rating Scale (MADRS) score, and
b) orally administering an immediate release oral dosage form comprising 60 mg of esketamine or a pharmaceutically acceptable salt thereof, to the subject that is determined to have a Montgomery-Åsberg Depression Rating Scale (MADRS) score of at least 28; and
wherein the administration is once daily for at least 28 days.
